(19) United States
(12) Patent Application Publication
(10) Pub. No.: US 2012/0121774 A1
(43) Pub. Date: May 17, 2012

(54) SYSTEM AND METHOD FOR COATING BULK ARTICLES

(76) Inventors: James Marjeram, Markham (CA); David O'Hara, Toronto (CA)

(21) Appl. No.: 13/127,191

(22) PCT Filed: Nov. 12, 2010
(86) PCT No.: PCT/CA10/01756

§ 371 Date: May 2, 2011

(51) Int. Cl. B05C 11/00; A61J 3/00; A23G 3/54; A23P 1/02; A61K 9/28; A23P 1/08

(52) U.S. Cl. 426/302; 118/695; 427/2.14

(57) ABSTRACT

A system for coating to tablets or other articles includes an elongate pan for retaining a bed of articles, a coating discharge system comprising a plurality of individually-controlled spray zones located linearly along said pan, and a means for distributing said articles along the pan wherein the full surface of all of said articles is exposed to said spray. A controller is programmed to operate the system to initiate a batch mode start-up sequence to coat an initial load of articles, a continuous, or a shut-down sequence, by controlling the dispensing of coating fluid on a zonal basis, and optionally controlling the distribution of articles in the pan and their discharge by controlling the effective height of a weir and the degree of tilt of the system.

### FIELD OF THE INVENTION

The invention relates to equipment, systems and methods for coating articles in bulk, such as pharmaceutical or candy tablets, with a fluid-based coating. More specifically, the invention relates to coating articles by dispensing the coating fluid from overhead spray nozzles onto a bed of the articles placed in a pan, drum or the like.

### BACKGROUND OF THE INVENTION

Systems for coating articles such as edible tablets typically dispense a pressurized and atomized coating fluid onto a bed of the articles, for example a volatile liquid in which a coating composition has been dissolved or suspended. The coating fluid is dispensed through one or more spray nozzles onto a bed of articles that has been placed on a pan, which may comprise a rotatable drum or the like. In order to fully coat the articles, the bed may be agitated, churned or otherwise displaced in order to expose all of the articles within the bed to the coating spray. For example, the pan may be rapidly reciprocated or, in the case of a rotatable drum, rotated so as to tumble the bed and fully expose all surfaces of the articles to the spray nozzles. The volatile component of the coating fluid is evaporated, for example by directing a flow of heated air onto or through the bed. For this purpose, the pan or drum may include perforations to permit air flow therethrough. The system may be enclosed within a housing such as a cabinet to prevent unwanted discharge of gases, particulates and other by-products of the coating process into the environment. Coating systems may be configured to operate in a batch mode or, according to more recent systems, a continuous mode or a selected one of a batch or continuous mode. For the latter, an elongate pan or drum is provided, with the articles being dispensed onto a first end of the pan. The articles are caused to travel towards the opposed end, for example by rotating or otherwise moving the pan or drum, whilst continuing to introduce uncoated articles into the first end of the pan or drum. As the articles travel lengthwise down the pan, the coating fluid is dispensed onto the bed of bulk articles. The coating solidifies onto the individual tablets, and the fully coated articles are then being discharged from the opposing end of the pan or drum.

An important goal of a coating system, in particular one intended for coating pharmaceutical products, is uniformity and the ability to control the coating thickness, as determined by the tablet weight gain. As well, the articles should not be processed with undue roughness so as to minimize breakage, chipping and other damage to the articles while being treated. It is also desirable to minimize the wastage of product during the coating process, for example by minimizing or eliminating the presence of uncoated products during the start-up and shut-down phases of a coating operation, in particular for continuous coating processes and systems.

A continuous coating system is useful for large-scale operations, as it is capable of providing high capacity and efficient treatment of articles. However, it can be difficult to provide a continuous coating process which operates with a minimum of waste during the start-up and shut-down procedures. The consistency and uniformity of the coating can be difficult to maintain during start-up and shut-down of a continuous coater. One solution has been to provide a coating system which is capable of operating in a “batch” mode during start-up, subsequently switching to a “continuous” mode for the duration of the run. However, there is still a need for improvements in terms of efficiency and reduced waste generation in such systems.

An example of a continuous coating system is described in the present inventors' previous patent application Ser. No. 11/911,498, the contents of which are incorporated herein by reference.

### SUMMARY OF THE INVENTION

According to one aspect, the invention relates to an apparatus for coating bulk articles such as edible tablets, which comprises the following elements:

a) an elongate pan, which may housed within a cabinet, for retaining a bed of said articles, with the pan comprising a first end for receiving uncoated articles and an opposed second end for discharging coated articles,

b) a coating delivery system for delivering coating onto a bed of articles within the pan, with the coating being delivered in a series of independently controlled zones which are distributed lengthwise along said pan,

c) a feeder to introduce articles into said pan on either of a continuous or batch basis,

d) means to distribute articles within the pan to expose them to the coating and to convey the articles from the first end to the second end of the pan, and

e) a controller in operative communication with said coating delivery system, feeder and means to distribute articles. The controller operates the apparatus in a batch mode for start-up or shut-down sequences wherein said coating delivery system is controlled to deliver different amounts of coating in respective ones of said zones to a bed of articles on said pan. The amount of coating delivered during the start up sequence is selected to substantially fully coat only the articles located in the zone at the second end, while the amount of coating delivered in said shut down sequence is selected to substantially fully coat the full bed of articles. The controller also operates the system in a continuous mode wherein articles are introduced by the feeder on a continuous basis into the first end and discharged from the second end while said coating is delivered to the articles within all of said zones on a continuous basis.

The coating dispensing system may be adapted for discharging coating in the form of a spray onto a bed of the articles. Preferably, the coating is discharged in the form of a liquid which contains a coating suspended or dissolved therein, but it is contemplated that other forms of a coating discharge may be employed. The discharged coating spray is referred to herein as a “coating fluid”, which is broadly defined to include a coating substance contained in any suitable carrier which may be applied to the articles. The dispensing system includes a source of pressurized coating fluid. The system may comprise a linear array of spray nozzles within the cabinet, connected by a conduit to the fluid source, in which the independent control is achieved by controlling air pressure and/or the flow of coating liquid to the nozzles.

The invention may further include a cabinet support structure for supporting the cabinet and selectively tilting the cabinet along its elongate axis between a first position wherein the pan is substantially horizontal and a second position wherein said pan declines downwardly towards said second end, as well as intermediate positions. The support structure is associated with an actuator which effects the tilt by selectively elevating or lowering an end of the cabinet.

The pan may comprise a drum journalled for rotation within said cabinet, said apparatus further comprising drive means to rotate said drum, wherein said means to distribute the bed of articles comprises rotation of said drum.

In another aspect, the apparatus includes a weir located at the distal end of the drum to retain said articles within said drum at a selected bed depth. The weir is mounted to the cabinet to remain static during rotation of the drum, such that it maintains its position at the base of the drum as the drum is rotated, thereby forming a fixed-position dam for retaining a bed of articles of a selected height within the drum. During operation on a continuous basis, articles within drum spill over the weir as over a dam, when the bed depth exceeds the effective weir height. The weir is generally crescent-shaped with its lower edge being semi-circular with a radius essentially equal to the drum radius. The weir is shaped as a segment of a cone, to project outwardly from the drum. The position of the weir relative to the drum may be adjusted by rotating the weir about an axis which is co-axial with the elongate axis of the drum, thereby adjusting the effective height of the weir relative to the drum. When the position of the weir is adjusted into a position adjacent to the base of the drum, it effectively has a maximal spill-over height relative to articles in the drum. As the weir is rotated away from this position, its effective spill-over height decreases so as to lower the height of the bed retained by the weir. The weir can be rotated into a selected rotational position and then fixed in said selected position relative to said cabinet to retain a bed having a selected depth within the drum. The weir may be rotated into a non-obstructive position in which the weir does not present any obstacle to the discharge of tablets from the drum. The weir configuration provides an inside surface of the weir, facing the drum interior, which slopes outwardly. The slope of this surface assists in the discharge of articles from the drum, and minimizes the risk of articles becoming lodged in the junction between the drum and weir.

The apparatus may further comprise an array of individually controllable heat sources for delivering heated gas into said cabinet and defining discrete heating zones disposed linearly along the length of said cabinet for drying and/or curing said articles, said heat sources being in operative communication with and responsive to said controller for selectively applying a heated airflow within selected ones of said zones. A plurality of bypass valves and bypass ducts may be associated with said heat sources and controlled by said controller, in a manner wherein shutting off selected ones of said heat sources directs the airflow from said heat source into corresponding ones of said bypass ducts without entering into said cabinet.

According to another aspect, the invention relates to a method for applying a coating to tablets or other articles in a bulk process, comprising the steps of:

a) providing an apparatus comprising an elongate pan for retaining a bed of said articles, said pan comprising opposed first and second ends, a coating delivery system for dispensing coating onto said articles in a plurality of individually-controlled spray zones located linearly along said pan, and a distribution system for distributing said articles along the pan wherein the full surface of all of said articles is exposed to said coating; and

b) selectively operating the apparatus in one of i) a batch mode for a start-up or shut-down sequence wherein said coating delivery system is controlled to deliver different amounts of coating in respective ones of said zones to a bed of articles on said pan, or ii) a continuous mode wherein articles are introduced on a continuous basis into the first end of the pan and discharged from the second end of the pan while applying spray within all of said zones on a continuous basis. The amount of coating delivered in said start up sequence is selected to substantially fully coat only the articles located in the zone at said second end, while the amount of coating delivered in said shut down sequence is selected to substantially fully coat the full bed of said articles.

Preferably, the delivery of different amounts of coating comprises delivering said spray for different durations within said zones. The start-up sequence may comprise sequentially actuating said spray zones for delivery of spray for increasingly longer durations of coating delivery from the first end of the pan to the second end of the pan, and optionally providing a duration during which all of said spray zones are actuated. The shut-down sequence may comprise sequentially actuating said spray zones for delivery of spray to provide increasingly shorter durations of coating delivery from the first end of the pan to the second end of the pan to fully coat all of the articles in the pan, followed by discharge of the articles from the pan.

In another aspect, the pan is tiltable along its elongate axis to selectively decline towards its second end, and the controller is configured to operate the apparatus according to the following sequence:

(a) a loading stage wherein said cabinet declines, and said pan is charged with a load of articles for coating on a batch basis

(b) a start-up sequence wherein said cabinet is horizontal, distributing means are activated so as to provide even exposure of the bed to the coating, and said coating is dispensed onto the articles in the pan in a zone by zone sequence. This initial stage is followed by a stage during which coating is dispensed within all zones. Each zone is activated after a selected duration from the previous zone, such that articles receive progressively more coating from the second end of the pan to the first end. At the conclusion of the start-up sequence, articles located in the zone at the second end are fully coated and ready for discharge while the articles located upstream of the second end are progressively less coated and require progressively larger doses of coating before discharge. The duration of each stage in the sequence is selected such that when the start-up stage is complete and the delivery system is activated in all zones, the articles are fully coated upon discharge from the pan.

(c) a continuous coating sequence wherein a continuous stream of articles is dispensed into the pan, the distribution means is activated, and said cabinet declines to assist in the movement of articles from the first end to the second end, and their discharge from the pan. The coating is dispensed in this stage within all zones along the full length of said pan.

(d) optionally, a batch mode shut-down sequence is provided wherein the delivery of articles to the pan and dispensing of fluid are halted, and the cabinet is tilted back into the horizontal position. This is followed by activation of the distribution system to expose articles in the pan to the coating fluid. Fluid is dispensed in a zone by zone sequence which is essentially the reverse of the start-up sequence, such that articles in the zone at the second end of the pan (which have already been exposed to the coating spray for a longer duration than articles in the preceding zones) receive the shortest duration of the coating spray, and the articles at the first end receive the longest duration. The duration of each step in the sequence is selected such that by the end of the shut-down sequence, all articles in the pan have become fully coated. The shut down stage concludes by tilting the pan to discharge the load of coated tablets from the pan.

Optionally, the pan may be tilted into a third, more steeply pitched position, for more effectively distributing articles within the pan at the commencement of the start-up stage and/or discharging the articles in the final step of the shut down stage.

In a further aspect, the controller controls the position of the weir described above, so as to control the bed depth during the above steps. The weir is positioned to partially obstruct the pan by a selected amount during all steps, except for the last step of the optional shut-down stage in which the articles are fully discharged from the pan, when the weir may be positioned in a non-obstructive position.

The apparatus may further comprise an array of heat sources for delivering heated gas into said cabinet for contacting said articles, said array comprising a plurality of heating zones disposed linearly along the length of said cabinet. The zones are substantially equivalent in position to the zones defined by the coating delivery system. The heat sources are in operative communication with and responsive to said controller for selectively and sequentially applying a heated airflow to selected zones disposed lengthwise along said pan. The heat sources each may include a bypass duct and a bypass valve, wherein when said heat sources are shut off, airflow from the heat source passes into said bypass duct and is not directed into the interior of said cabinet. The controller may be programmed to control the operation of the heat sources and bypass ducts, such that heated air is delivered into the cabinet.

Preferably, the apparatus and method described herein provides a low or essentially zero waste mode of operation. Reduction of waste is enhanced when the weir comprises the semi-conical configuration described herein. A semi-conical dam having a similar configuration may also be provided at the first end of the drum-shaped pan to further reduce wastage. A further aspect which reduces waste is a shut-off valve associated with the tablet-feeding device, which is located adjacent to the pan so as to minimize the number of tablets which may become lodged in the tablet feeder.

In the present specification, the use of directional references such as “horizontal”, “vertical”, and the like, are understood to include departures from absolute values except where the context otherwise makes clear. As well, dimensions and other similar parameters and values are understood to represent merely examples of the invention and are not intended to limit the scope of the invention except where specifically stated to do so. The detailed description presented below is intended merely to illustrate one embodiment of the invention and is not intended to limit the scope thereof.

The terms “articles” and “tablets” refers broadly to relatively small articles suitable for coating in bulk in an industrial operation, by tumbling or churning a bed of such articles while exposing them to a spray. Suitable articles include without limitation pharmaceutical or other edible tablets, pills, lozenges, tablets, particles and the like. It will be understood that these terms may be used interchangeably, and are intended to have a broad meaning except where specifically indicated otherwise.

### BRIEF DESCRIPTION OF THE DRAWINGS

FIG. 1 is an isometric view of an apparatus according to the present invention, wherein the exterior panels of the cabinet have been removed to show internal components.

FIG. 2 is a side elevational view of the apparatus of FIG. 1.

FIG. 3 is a perspective view of a portion of the apparatus.

FIG. 4 is a perspective view of the rear port of the apparatus.

FIG. 5 is an elevational view of the rear port.

FIG. 6 is an elevational view of the weir portion of the apparatus.

FIG. 7 is a perspective view of the weir.

FIG. 8 is a side elevational view of the rear port

FIG. 9 is a perspective view of the rear port, showing the weir plate therein.

FIG. 10 is a perspective view of the rear port.

FIG. 11 is a perspective view of the front (proximal) port.

FIG. 12 is a further perspective view of the front port.

FIG. 13 is a further perspective view of the front dam.

FIG. 14 is a side elevational view of the front dam.

FIG. 15 is a schematic view of the interior of the apparatus, showing in particular the spray nozzles and spray pattern generated thereby.

FIGS. 16a through 16f depicts a flow chart, showing operation of the present system.

FIGS. 17A through 17E are schematic diagrams of the apparatus, illustrating the system in its various modes of operation.

### DETAILED DESCRIPTION

Cabinet and Drum Structure

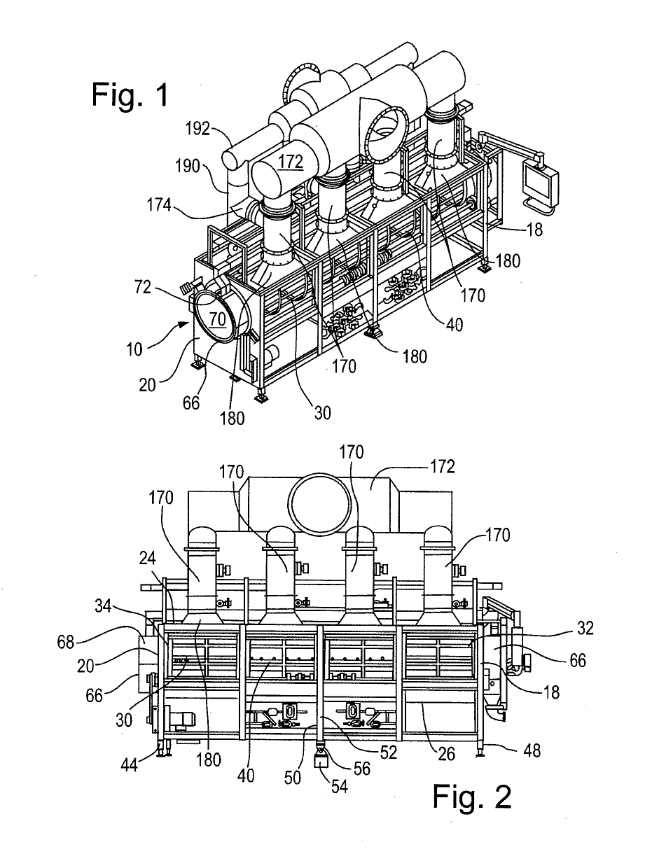

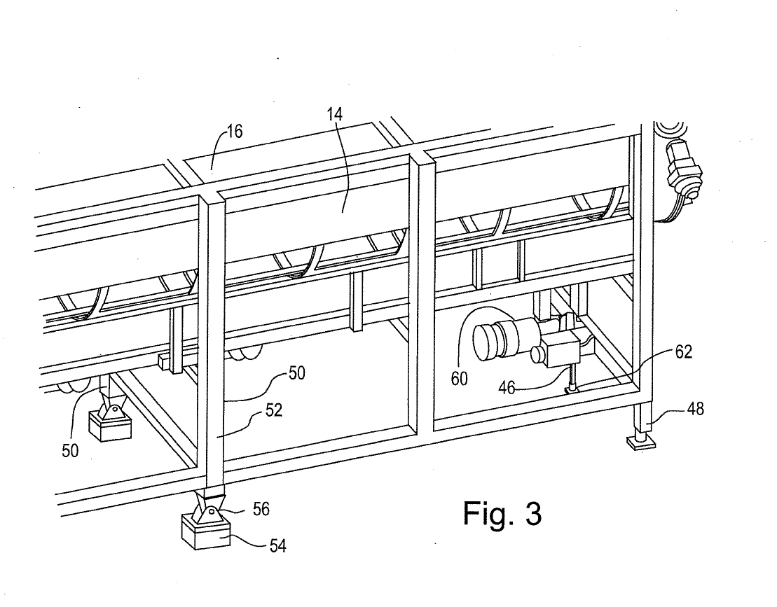

Turning to FIGS. 1-3, an elongate cabinet 10 is provided for housing certain of the system components. The cabinet 10 comprises a rigid frame with a skin composed of fixed and openable panels 14 and 16. In FIGS. 1 and 2, the cabinet 10 is shown as an open framework, with panels removed to show internal structure. The cabinet is effectively sealed when closed, whereby the interior of the cabinet forms a sealed environment to prevent escape of gases, particulates and the like from the cabinet interior under normal operating pressures, except via exhaust openings, as discussed below. The cabinet includes proximal and distal end walls 18, 20 with an elongate axis extending therebetween, vertical side walls 22 and horizontal top and bottom walls 24, 26.

A rotatable, horizontally-disposed drum 30 is housed within the cabinet 10, and extends between the end walls 18 and 20. Drum 30 forms a pan for supporting a tablet bed, as discussed below. Drum 30 extends lengthwise within cabinet 10 between end walls 18 and 20, and comprises proximal and distal ends 32, 34 with an elongate central axis extending therebetween. Drum 30 is journalled for rotation about its central axis. The drum comprises a rigid framework 40, which supports a perforated tubular drum wall, not shown. The perforations are of a size to effectively permit airflow through the drum wall, while retaining the tablets or other small articles to be coated within the drum. The perforation size is based on the expected use of the apparatus, for example the dimensions of the articles that may be processed in the drum. Drum 30 is rotatably driven by an electric motor or other speed-controllable drive means, operatively connected to drum 30 via a drive wheel, belt or other drive means, not shown. Rotation of drum 30 effectively distributes tablets within the drum and also “churns” the tablet bed to expose all surfaces of the tablets to overhead sprayers. Those skilled in the art will appreciate that these functions can be performed by distribution means other than a rotary drum, including imparting other forms of motion such as reciprocating motion.

Cabinet 10 is supported on an underlying surface by a support substructure comprising an array of legs, posts or similar supporting members. At the distal end, the support substructure comprises a pair of variable-length legs 44, located adjacent to the distal corners of the cabinet. Legs 44 are substantially non weight-bearing. At the proximal end, an extendible weight-bearing support 46 is provided, located at the midline of the cabinet. Additional variable-length non weight-bearing legs 48 are located at the proximal corners of the cabinet. A pair of fixed length weight-bearing intermediate legs 50 is located on opposing sides of the cabinet, intermediate between the proximal and distal legs. Intermediate legs 50 each comprise a downwardly-extending post 52, a ground-contacting foot 54, and a pivot joint 56 between the post and foot portions. The pivot joint permits the foot 54 to remain fixed to the ground surface, while permitting the post 52, and the cabinet 10 as a whole, to tilt in fore and aft directions.

The extendible support 46 at the proximal end constitutes a tube-in-tube telescoping member which can be selectively extended or retracted in an infinitely adjustable fashion. Extension and retraction of the support is effected by any suitable actuator 60, including a pneumatic or hydraulic ram, mechanical jack, or other known means to extend or retract an elongate supporting member of this nature. In the described embodiment, the support comprises a pneumatic ram fed by an air supply which is not shown, but is conventional. The support terminates in a weight-bearing foot 62, which is fixed to or rests upon the underlying surface. The pneumatic or other drive means 60 for the support is operatively linked to a central controller 100, described below, to cause the support to lengthen or retract in a controlled fashion.

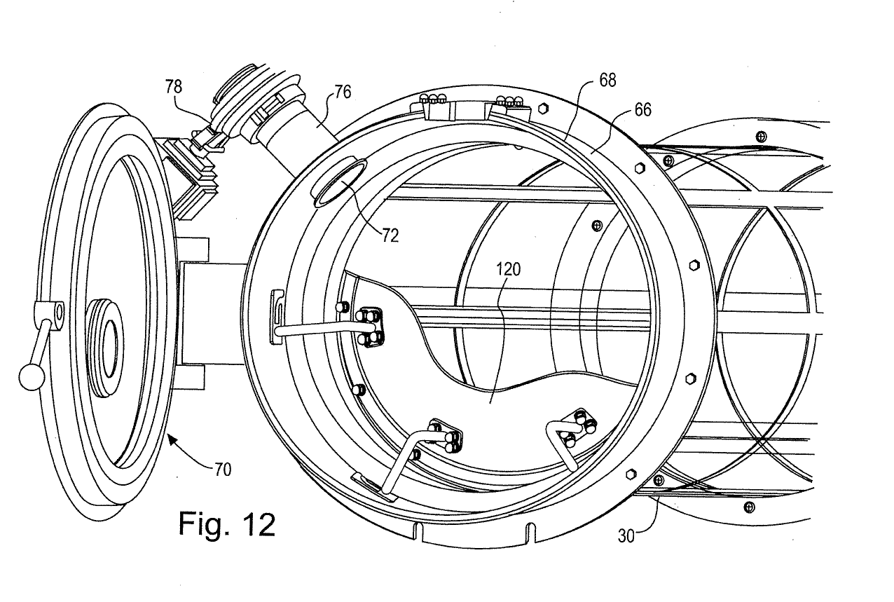

Cabinet 10 may be tilted in a fore or aft direction by extension or retraction of proximal support 46. Extension of support 46 elevates the front of the cabinet, as see in FIG. 12A, thereby tilting the cabinet downwardly towards the distal end of the cabinet. As will be described in detail below, the degree of tilt towards the distal end is selected to facilitate movement of the tablets or other articles within the drum, from the proximal to the distal end thereof. In the embodiment described herein, the cabinet may be tilted within a range from horizontal to declining by about 6 inches over the length of the cabinet, namely about 16-20 feet. As the cabinet tilts, the four corner legs elongate or retract, thereby maintaining contact with the ground. The corner legs are substantially non weight-bearing and are provided primarily to fence off the underside of the cabinet.

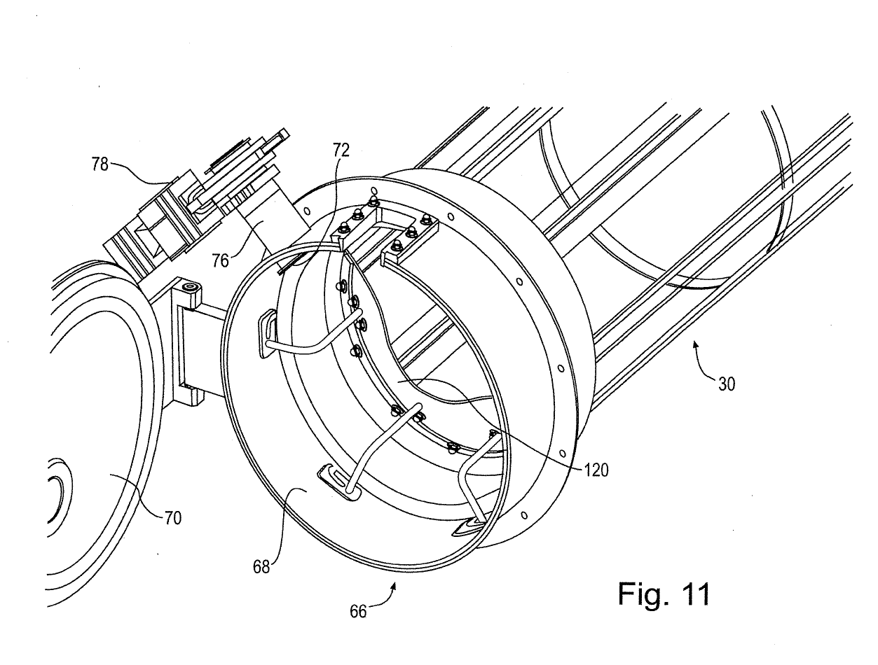

The proximal end wall 18 of the cabinet 10 includes a protruding cylindrical entry port 66, seen in detail in FIGS. 11 and 12. The interior of the entry port 66 communicates with the drum interior for introduction of uncoated articles into the interior of drum 30. Entry port 66 comprises a cylindrical wall 68 and a hinged end panel 70, which opens to permit cleaning, maintenance and other access to the interior of drum 30 and cabinet 10. Uncoated articles are introduced into the entry port through an opening 72 in the cylindrical wall. The articles are dispensed in a controlled fashion with an in-feed conveyor system (not shown), which is located above the level of drum 30 to permit the articles to be introduced into the drum by gravity feed. The in-feed conveyor is of a conventional type, for example as described in applicant's prior patent application Ser. No. 11/911,498. The uncoated articles are fed from the conveyor into the entry port through a duct 76, which communicates with an internal chute located within the inlet port to feed tablets directly into the rotatable drum.

A shut-off valve 78 is provided within the duct to selectively stop the flow of tablets into the drum. Valve 78 may be located at any convenient position along the duct, preferably adjacent to or close to the lower thereof where the duct enters the entry port. Valve 78 consists of a butterfly valve, which is responsive to the central controller. Shutting off valve 78 prevents stray particles from entering the housing once the shut-down sequence has been initiated, as will be discussed in detail below. A dam 120, described in more detail below, is mounted within the port 66 to retain the tablet bed within the drum.

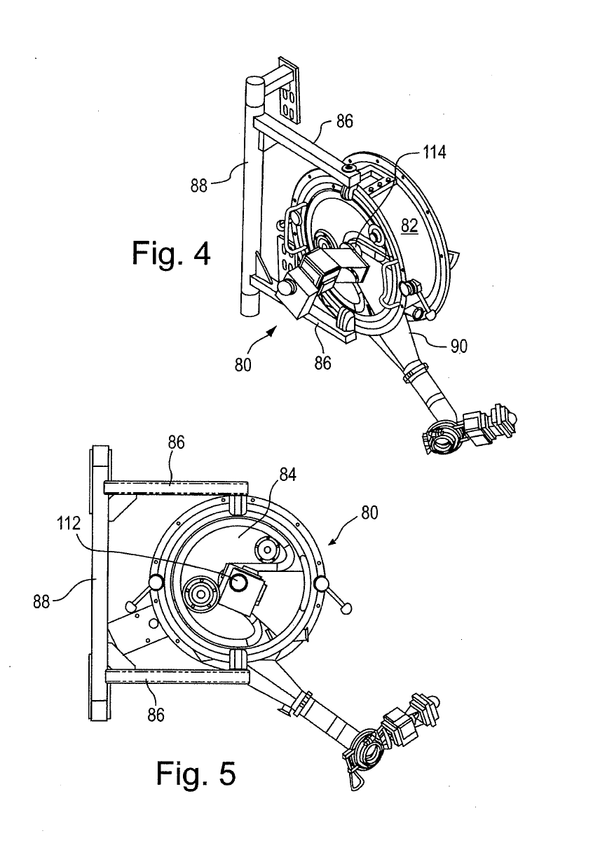

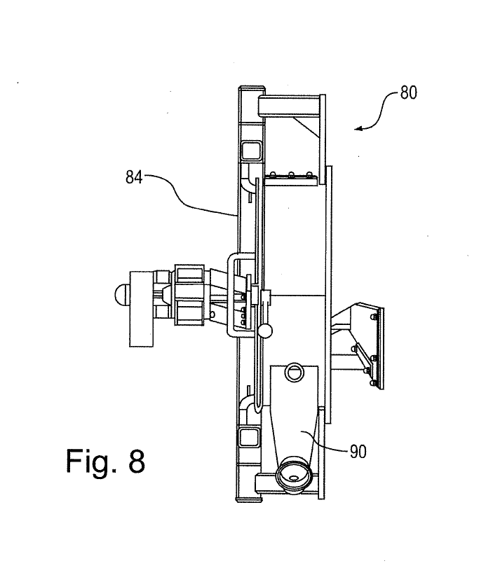

An outlet port 80, shown in detail in FIGS. 4-10, is provided at the distal end of the cabinet, having a similar protruding cylindrical configuration as the inlet port. The outlet port includes a cylindrical wall 82 and an openable panel 84 to provide access to the interior of the drum and cabinet. The panel 84 is hinged to the wall 82 via a hinge bracket composed of upper and lower horizontal arms 86a and b, which fasten to opposing upper and lower segments of the panel. The arms are hinged to the cabinet via a vertical hinge bar 88 which is mounted adjacent to a corner of the cabinet 10. The hinge bar 88 permits the arms 86 to swing outwardly about a vertical axis defined by the hinge bar, thereby swinging the panel outwardly away from the outlet port. When open, the panel is displaced at some remove from the cabinet to facilitate access. The door panel also includes a window to permit viewing of the interior of the drum and cabinet.

A discharge chute 90 extends from the lowermost portion of the outlet port 80. The chute 90 communicates with the interior of port 80, and is configured such that coated articles discharged from drum 30 into port 80 enter into the discharge chute for removal from the system. The discharged articles may be deposited into a suitable container, or alternatively into a downstream processing operation, for further processing, packaging or the like.

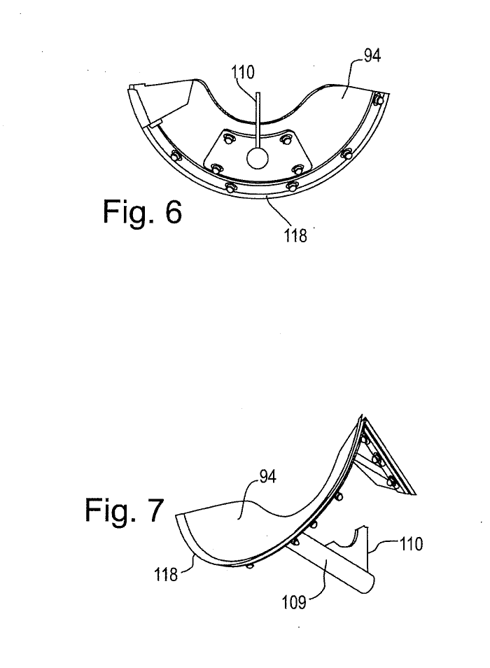

A rotatable weir plate 94 is disposed within outlet port 80. Weir plate 94, shown in more detail in FIGS. 6 through 9, comprises, in front elevational view, an arcuate, generally crescent-shaped structure. As best seen in FIGS. 7, 8 and 9, weir plate 94 is a partial cone, i.e. a radial segment of a hollow cone in which the apex has been removed, which is mounted to project outwardly from drum 30. The outer rim of weir 94 is a segment of a circle, the radius of which is essentially the same as the drum in order that the weir may conform to the configuration of drum 30. The inner surface 96 of weir plate 94 faces the interior of the drum and slopes outwardly from the drum interior. Weir plate 94 is mounted to the outlet port for rotation relative to the cabinet, wherein the weir plate may be selectively positioned within a range of rotational positions relative to the cabinet. During normal operation of the apparatus, weir plate 94 remains static in position as the drum rotates. The rotational position of weir plate 94 can be adjusted by rotating the weir plate about an axis which is co-axial with the central axis of drum 30. This position is selectively adjusted for operation of the system in its various modes, as will be described below, and can effectively serve to change the tablet bed depth within the drum. A mounting bracket extends from the rear surface of the weir plate, comprising a pair of offset horizontal shafts 109 and 111, linked by an arm 110. Arm 110 has an elongate axis which is perpendicular to shafts 109 and 111. Shaft 109 extends distally from weir plate 94. Shaft 111 extends through a sealed opening in rear panel 84, and has a central axis which is co-axial with the central axis of rotation of drum 30. Shaft 111 is journalled for rotation about is axis, thereby causing weir plate 94 to rotate in an arcuate motion about a circle defined by its geometric center, thereby permitting the weir plate to rotate relative to the drum 30 without any displacement of its outer circumferential edge relative to the drum surface.

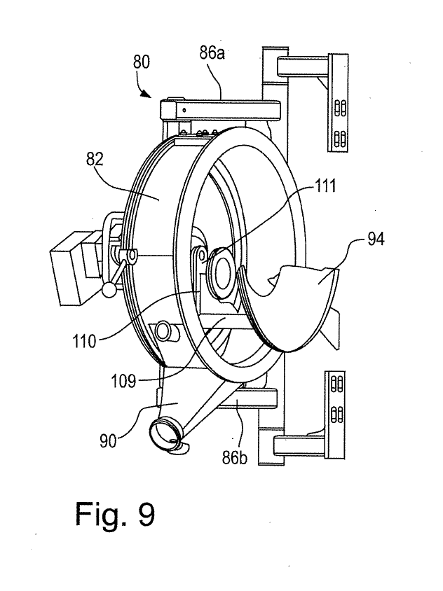

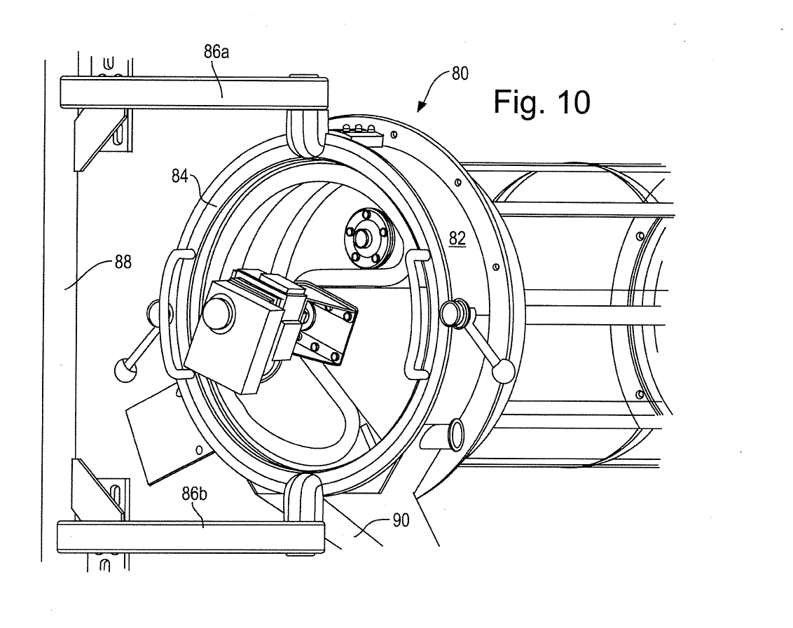

Weir plate 94 is positioned such that its outer rim 118, which is configured as a segment of a circle, is located slightly within the drum interior, or in contact with the rear rim of drum 30. In a lowermost position, as seen in FIG. 9, the rim of weir plate 94 corresponds in position to the lowermost segment of the drum wall. Weir plate 94 may be rotated about the drum axis in either direction, thereby effectively changing its spill-over height relative to the tablet bed within drum 30. Rim 118 is formed of a low-friction material such as Teflon™, to avoid drag and wear as the drum surface moves relative to the stationary weir plate 94.

As is seen in FIG. 9, weir plate 94 effectively protrudes in a distal direction. The sloping inside surface 96 of weir plate 94 is configured such that the free circulation or “upwelling” of tablets within the bed will be maintained as the bed is “churned” upon rotation of drum 30. This circulation of tablets occurs even for tablets which travel to the region where weir plate 94 and drum 30 intersect. This sloping surface may avoid a drawback present in prior art structures, wherein tablets could become lodged where the weir plate meets the drum wall. Such tablets become effectively trapped at the base of the weir plate, and are thus not fully exposed to the coating spray. In contrast, the present sloping design encourages circulation of the tablets as the drum is rotated, such that a reduced portion of the tablets become trapped at the juncture between the weir plate and drum and remain uncoated. With the outwardly-sloping structure of the weir plate, all or substantially all of the tablets become exposed to the coating spray as the drum rotates.

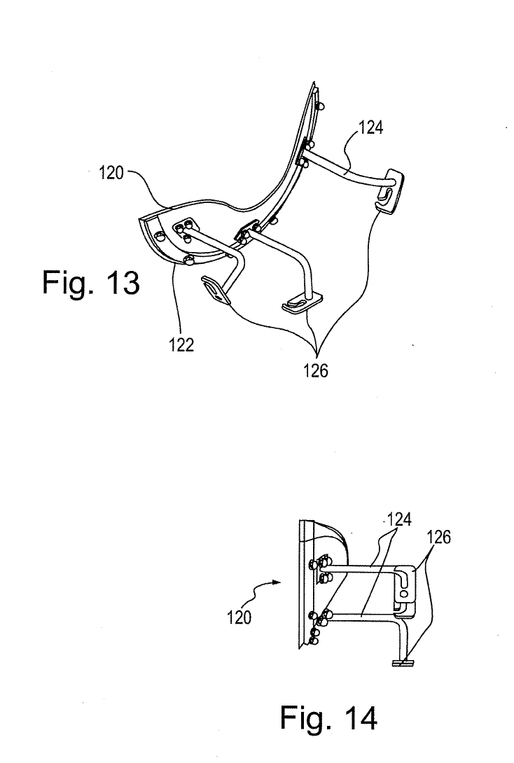

A tablet retainer dam 12, shown in detail in FIGS. 13 and 14, is mounted to the system at the proximal end of the drum 30 at a location and in configuration opposed to weir plate 94. Dam 120 effectively comprises a mirror image of weir plate 94 but is mounted in a fixed, non-rotatable position relative to the cabinet. Dam 120 is located partially within the drum interior, in a fashion similar to the weir plate, and projects outwardly from the drum in a proximal direction. Dam 120 contacts the interior surface of the drum in similar fashion to the distal weir plate, and is likewise provided with a low-friction outer surface 122 where the rim of the dam contacts the drum interior. Dam 120 is configured to retain a bed of tablets within the drum interior in a non-adjustable fashion.

Zonal Spray System

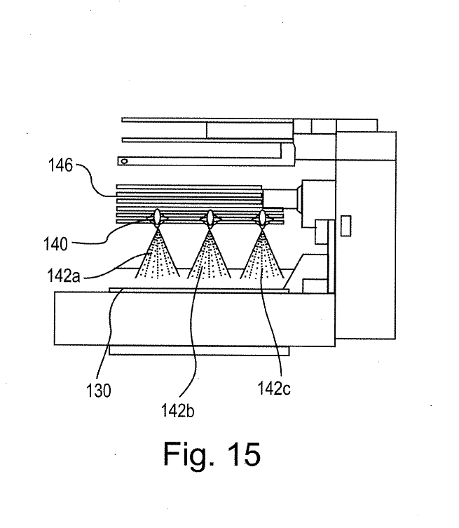

As shown in FIG. 15, coating liquid is dispensed onto a tablet bed 130 within drum 30 via an array of spray nozzles 140. Nozzles 140 are independently controlled to dispense coating fluid, in atomized form, in a series of spray zones 142a, b and c such that delivery of fluid within each zone is independently controlled, as described in more detail below. The nozzle array is supported an elongate rack 146 that during use is located within the interior of the drum 30. Rack 146, which is of the type described in U.S. patent application Ser. No. 11/911,498, can be removed from drum 30 for cleaning, maintenance, or the like.

An array of spray nozzles 140 is mounted to each support. The nozzles comprise any suitable nozzle or spray delivery means for spraying a liquid onto the tablet bed. In the present embodiment, the nozzles comprise Schlick™ spray nozzles, configured to deliver an atomized coating spray. The spray nozzles are removable for cleaning, maintenance, etc. A supply of coating fluid and air (or other gas) to generate an atomized spray is fed to the nozzles through hoses, not shown, which deliver a controlled amount of fluid to the nozzles under pressure. The rate of delivery of the coating liquid and pressurized air to the nozzles is controlled by valves, which are responsive to the central controller. Each valve controls the flow to an individual sprayer or sprayer pair with the operation of each valve being independently controlled by the central controller. As seen schematically in FIG. 15, the nozzles are configured to discharge fluid either individually or as a pair. The fluid is discharged onto bed 130 as a cone 142 with minimal overlap between adjacent spray cones to provide a zonal spray pattern defined by the sprayers. The spray cones cover the full length of the tablet bed. The spray nozzles may be arranged in side-by-side pairs for complete coverage of the bed width.

The spray discharge pattern defines a series of zones 142a, b and c in a linear array within drum 30. The zones are substantially non-overlapping. The system is configured to selectively deliver spray to the tablet bed in an adjustable fashion, with spray being delivered to all of the zones or selected ones of said zones.

Zonal Heating System

As seen in FIGS. 1 and 2, an array of hot air supply ducts 170 discharge heated air into the cabinet in a series of heating zones along the length of the cabinet. As seen in FIG. 1, four such ducts 170 are provided, although any number of supply ducts, and corresponding heating zones, may be provided depending on the length of the cabinet and the desired length of each zone. The heating zones may correspond to the spray zones, or alternatively a lesser or greater number of heating zones may be provided relative to the spray zones. Supply ducts 170 connect to a common manifold 172 that supplies a pressurized stream of heated air. The pressure, temperature and other parameters of the heated air supply are determined by the required parameters of the coating operation which the apparatus is required to perform. Each supply duct 170 is joined to a bypass shunt duct 174 located upstream from the cabinet. The supply ducts are provided with a bypass valve 176 which controls airflow through the duct, with the valves being responsive to the central controller. When a duct is in the bypass mode, the valve shuts off the supply of heated air through the supply duct and shunts the airflow into the shunt duct, which discharges into the exhaust manifold, described below. In this fashion, a constant airflow is maintained through the non-bypassed ducts. Ducts 170 each include a flared lower segment 180 which opens into the cabinet 10. Ducts 170 are arranged to provide a flow of heated air into the cabinet interior along the full length of the cabinet. Preferably, the ducts are aligned with the spray discharge ones defined by the locations of the spray nozzles, such that full control over the spray discharge and heating can be controlled in a zonal fashion wherein both spraying and heating are performed within a series of discrete zones along the length of the cabinet.

Airflow is maintained through drum 30 by providing a configuration wherein the heated airflow passes through the perforations in the wall of drum 30, passing over or at least partially through the tablet bed, and into the lower portion of the cabinet. The airflow is then fed into an array of exhaust ducts, which lead into a common plenum for discharge to an air treatment system, not shown.

Control System

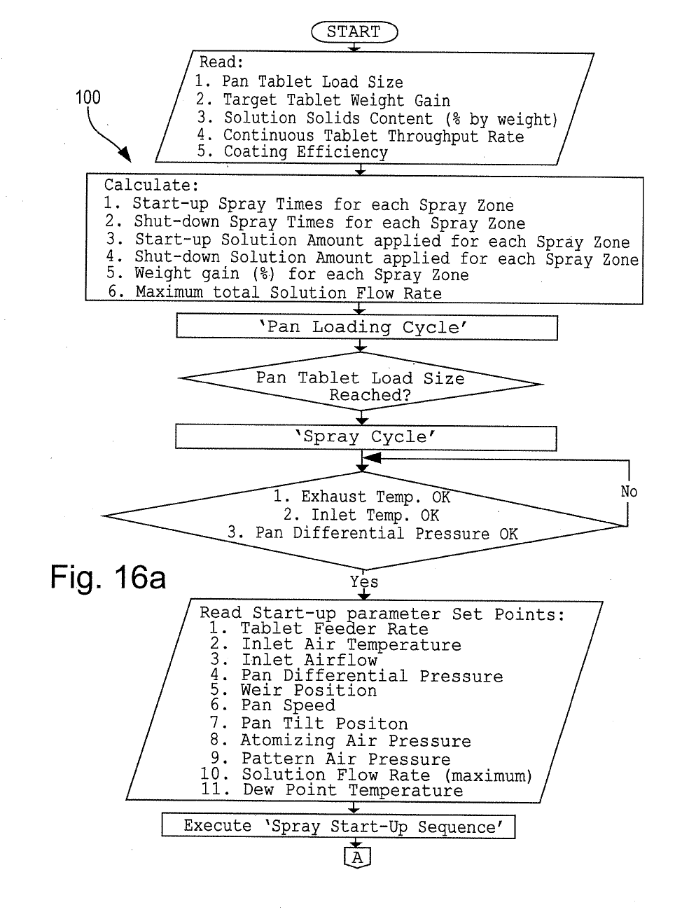

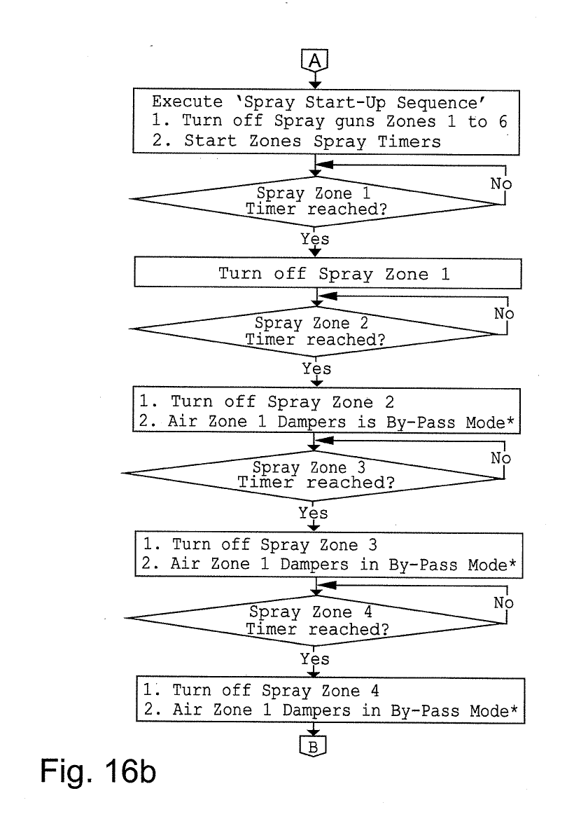

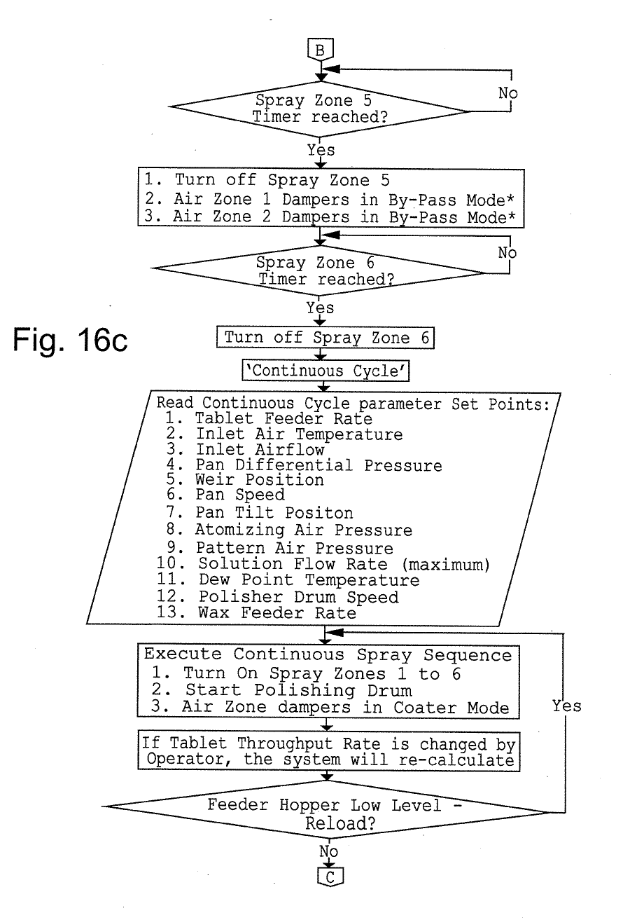

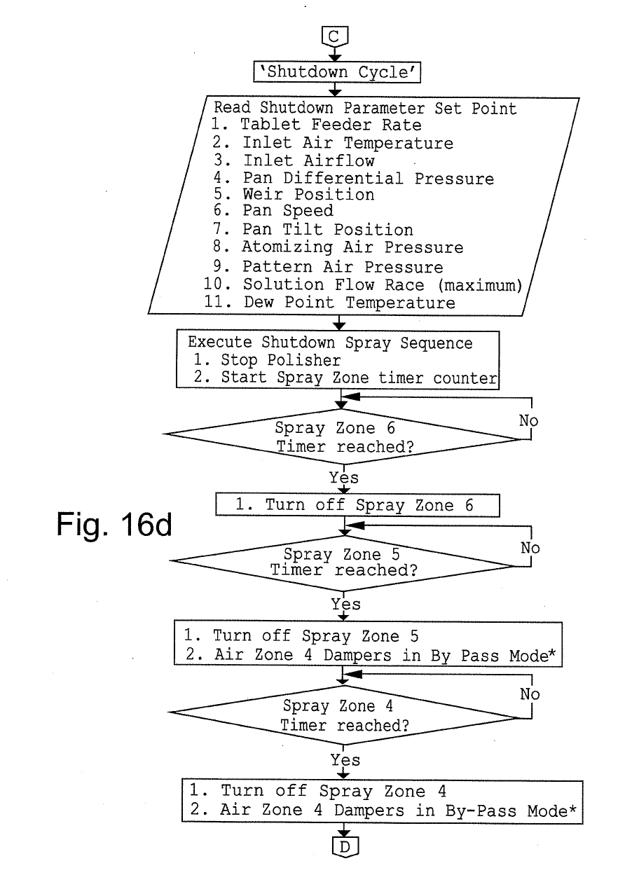

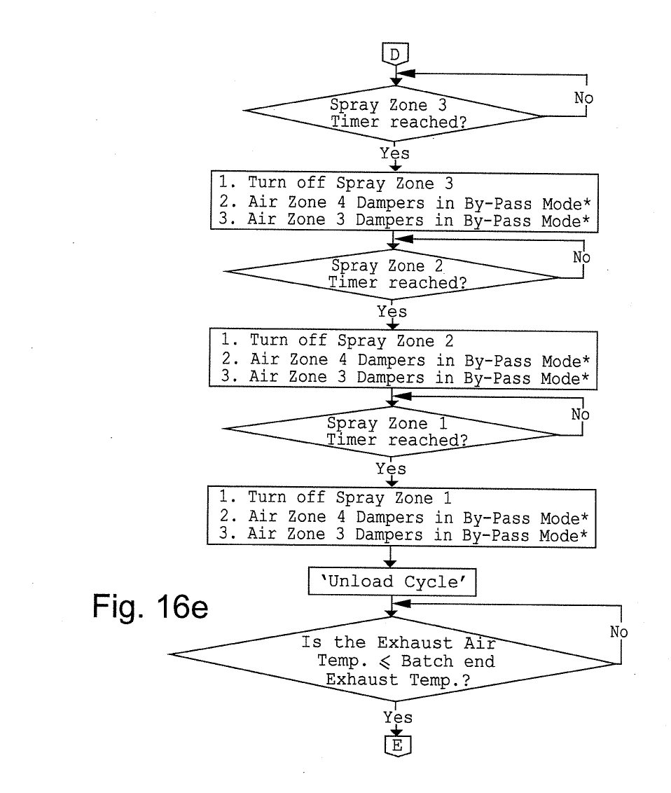

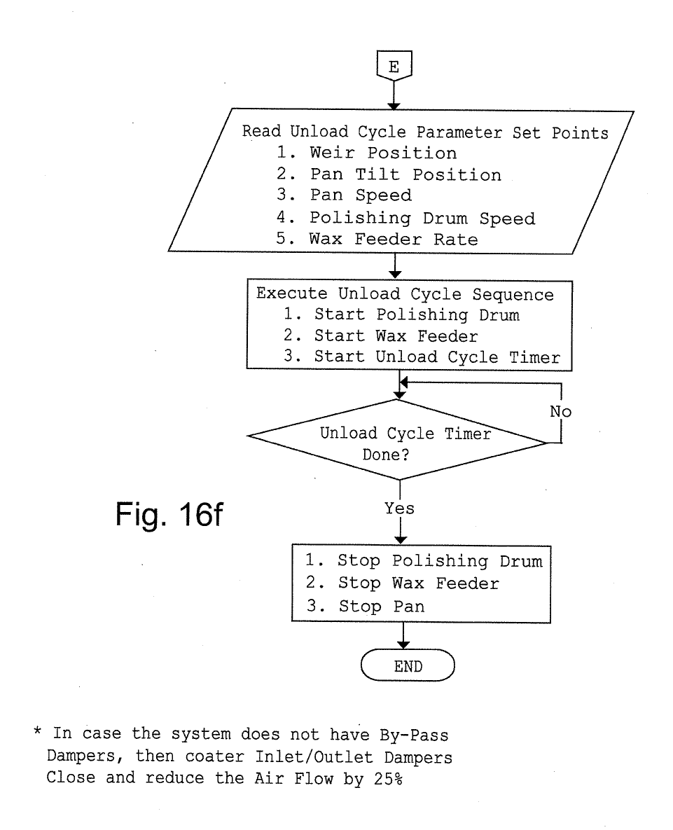

As described by way of a flow chart in FIGS. 16a-f, operation of the apparatus is controlled by a central controller 100, which may comprise a personal computer (PC) that includes a computer program medium to configure controller 100 to control the various components of the apparatus as well as associated components. Controller 100 receives and transmits signals to the system components and associated components via any convenient signal transmission means including an optical or conventional cable, wireless transmission, or any suitable form of data transmission line or means. Controller 100 is initially provided with predetermined system parameters including the optimal or desired load size of tablets within the pan or drum (which may differ depending on whether the system is operating in a batch or continuous mode), the target weight gain of the tablets resulting from the coating operation, the solids content of the coating solution, expressed as a percentage of solids by weight of solution, the target tablet throughput rate through the system, and the target coating efficiency determined as a percentage of the total dispensed coating material that adheres to the tablets.

The controller is also supplied with data reflective of operating conditions which is predetermined based on prior experimentation or other means of determining such operation data. These operating conditions includes:
(a) the target duration of the spray time during the start-up and shut-down modes for each spray zone
(b) total volume of coating solution applied within each spray zone, during the start-up and shut-down modes;
(c) the target of the tablet weight gain, within each spray zone during system operation;
(d) maximum flow rate for the coating solution during system operation.

The controller also receives operational data on a real-time, ongoing basis, including the following:
(a) tablet feeder rate;
(b) inlet air temperature;
(c) inlet air flow rate;
(d) pan differential pressure;
(e) rotational position of the weir;
(f) rotational speed of the drum;
(g) angular declination of the drum and cabinet;
(h) atomizing air pressure, for the air supply fed to the spray nozzles;
(i) pattern air pressure;
(j) maximum flow rate of coating solution;
(k) Dew point temperature.

The controller is configured to operate the system components, based on at least the above information, according to the flow chart illustrated in FIGS. 16a-f. In response to the inputs described above, the controller controls operating parameters of the system including:
(a) The rate of infeed of the tablets
(b) Air pressure to the sprayers for on/off control and modulation of spray rate
(c) Coating composition delivery rate to the sprayers
(d) Airflow and temperature of the hot air supply, including control of the bypass ducts
(e) Degree of tilt of the cabinet

During the start-up and shut-down modes, the controller controls operation of the tablet shut-off valve and sequentially controls the operation of the spray nozzles and hot air ducts to effect a sequential delivery of these inputs in a zone-by-zone fashion.

Persons skilled in the art will understand that programming of the controller can be carried out according to known methodology, in a generally conventional fashion, to effect the mode of operation described herein, including the flow chart of FIGS. 16a-f.

Operation in a “Zero Waste” Mode”

The system described herein is configured to operate according to a “zero waste” mode of operation, wherein there is minimal or essentially zero wastage of tablets, in particular during start-up and shut-down sequences. Wastage of tablets normally results from uncoated or partially coated tablets, which must be separated out and discarded, resulting in additional costs and environmental consequences. It will be understood that the term “zero waste” includes the generation of minimal wastes. One aspect of the “zero waste” mode is provided by a combination of the configuration of the weir plate 94 and opposed retainer plate 120 having a similar configuration, wherein the opposed plates slope outwardly away from the drum interior in opposing directions. This configuration facilitates circulation of tablets within the drum, where the tablet bed 130 comes into contact with the respective plates, thereby exposing all or essentially all tablets at the distal end to the coating spray. In contrast, it has been found that previous designs wherein the end plates are substantially vertical relative to the pan can result in some of the tablet bed becoming effectively lodged at the junction between the pan and weir plate, with such tablets not being exposed to the overhead spray and thus not becoming fully coated. As well, the sprayers are configured to discharge a cone of spray extending fully along the tablet bed, including where the tablet bed abuts the weir plate, thereby ensuring that tablets at this location are coated with the full measure of spray.

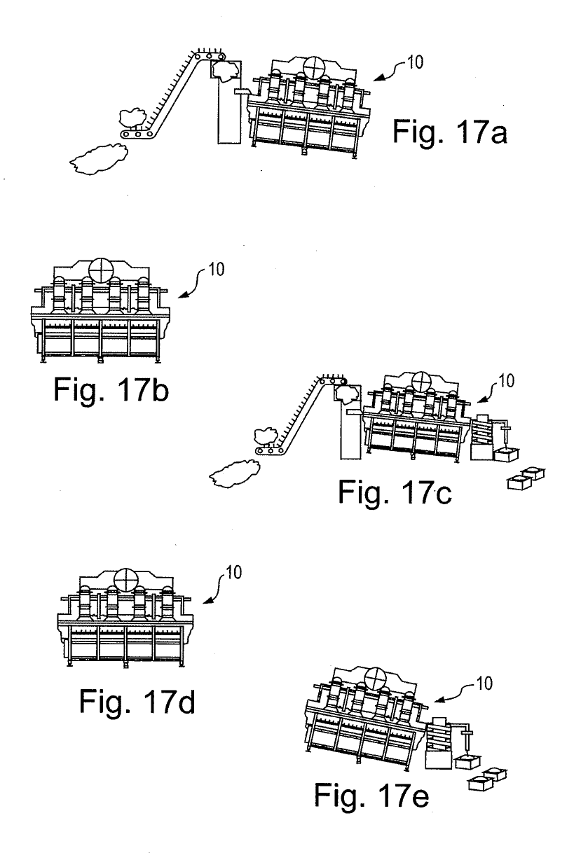

In addition, the “zero waste” mode involves operation of the system according to the start-up and shut-down sequences described below, and as depicted in the flow chart of FIGS. 16a through f and the schematic drawings in FIGS. 17a-e.

Start-Up Sequence

Prior to initiation to the start up sequence, operational data and parameters as described above are determined, and the operating conditions described above are calculated. The system is operated in a batch mode to coat an initial load of tablets during the start-up sequence.

a) Tilt cabinet 10 downwardly (which may comprise a maximally tilted position), position weir plate 94 for maximal spill-over height and commence rotation of the drum

b) charge drum 30 with an initial tablet load wherein the downward tilt and drum rotation are selected to distribute the initial load to form a bed of generally even depth along said the drum, with weir plate 94 continuing to block discharge from the second end of the drum,

c) level the cabinet/drum,

d) discharge coating liquid within all of said spay zones for a predetermined first duration to apply a partial coating to all of said initial load

e) sequentially deactivate the spray zones commencing at the first (inlet) end of the drum and terminating at the second (discharge) end wherein said spray is applied for progressively longer durations from the first end to the second end and articles at the second end become fully coated. The duration of each sequential deactivation is equal for all zones. At the conclusion of the start-up sequence, the drum is charged with a tablet bed wherein the tablets are fully coated in the spray zone at the discharge end and progressively less coated in zones towards the intake end. At this point, the continuous coating mode can be initiated.

Continuous Coating Mode

Following the start-up sequence, the system is operated in a continuous coating mode. For this stage:

a) the cabinet (including the drum) is tilted into an intermediate declining position to assist with tablet movement towards the discharge end, and the drum is rotated to tumble and distribute the tablet bed.

b) A continuous stream of uncoated tablets is fed into the drum at a selected rate.

c) The weir is position in an intermediate position selected to permit tablets to spill over the weir for discharge while maintaining a selected tablet bed depth

d) all of the spray zones are activated, whereby tablets discharged at from the drum are fully coated. The continuous mode is maintained for the full duration of the desired run. Optionally, the processing rate or other parameters may be adjusted during the run by any combination of adjusting the cabinet declination, tablet in-feed rate, spray rate, or weir position.

Shut-Down Sequence

a) the introduction of uncoated tablets at the first end is halted, and discharge from the drum is halted by positioning the weir for maximal spill-over height and elevating the cabinet to the horizontal position.

b) the spray zones are sequentially deactivated, commencing at the second (discharge) end and terminating at the first end. The sequential deactivation is carried out in a series of steps of equal duration for each zone. The total duration in which spray is dispensed within each zone is selected such that at the end of this stage, the entirety of the bed is fully coated. Rotation of the drum continues through this step, and throughout the shut-down sequence.

c) the tablets are discharged from said second end. This is affected by tilting the cabinet to its maximal downward position and positioning the weir in the non-obstructive position, whereby all of the tablets are discharged.

In the above sequences, the heating system may be controlled to deliver hot air into the cabinet within only the zones that are actively receiving spray discharge.

The selective declination of the drum, combined with control of the other variables described above, permits a highly efficient application of spray during the start-up and shut down sequences, as assisting in achieving the “zero waste” goal.

The foregoing has constituted a description of specific embodiments of the present invention. However, persons skilled in the art will understand that the foregoing description does not limit the full scope of the invention, but is intended merely to illustrate a particular mode or embodiment thereof. The full scope of the present invention will be apparent to those skilled in the art upon reading the present patent specification as a whole, including the claims. As well, it will be understood that certain of the features, elements and components described herein may be substituted for equivalents thereof, without substantially altering the nature or operation of the present invention. To the extent permissible, the inventors intend that all such equivalents form part of the present invention.

1. An apparatus for coating tablets or other bulk articles, comprising:
a) an elongate pan for retaining a bed of said articles, said pan comprising a first end for receiving uncoated articles and an opposed second end for discharging coated articles, said first and second ends defining an elongate axis therebetween;
b) a coating delivery system for delivering coating onto articles within said pan wherein said coating is delivered in a sequence of independently controlled zones which are distributed lengthwise in a linear sequence along said pan;
c) a feeder to introduce articles into said pan on a continuous or batch basis;
d) means to distribute articles within the pan to expose said articles to said coating and to convey said articles from the first end to the second end of the pan; and
e) a controller in operative communication with said coating delivery system, feeder and means to distribute articles, said controller being configured to selectively operate the apparatus in either of: i) a batch mode for a start-up or shut-down sequence wherein said coating delivery system is controlled to deliver different amounts of coating in respective ones of said zones to a bed of articles on said pan, wherein the amount of coating delivered in said start up sequence is selected to substantially fully coat only the articles located in the zone located at said second end and the amount of coating delivered in said shut down sequence is selected to substantially fully coat the full bed of said articles; or ii) a continuous mode wherein articles are introduced by the feeder on a continuous basis into the first end and discharged from the second end while said coating is delivered to the articles within all of said zones on a continuous basis.

2. The apparatus of claim 1, wherein said delivery of different amounts of coating comprises delivering said spray for different durations within said zones.

3. The apparatus of claim 1, wherein said controller is configured to provide said start-up sequence by sequentially actuating said spray zones for delivery of spray for increasingly longer durations of coating delivery from the first end of the pan to the second end of the pan, and optionally providing a duration during which all of said spray zones are actuated; and said controller is configured to provide said shut-down sequence by sequentially actuating said spray zones for delivery of spray to provide increasingly shorter durations of coating delivery from the first end of the pan to the second end of the pan to fully coat all of the articles in the pan, followed by discharge of the articles from the pan.

4. (canceled)

5. The apparatus of claim 1 further comprising a weir adjustable in position relative to said pan at said second end to retain a bed of selected depth of said articles within the pan, wherein said controller is further configured to adjust said weir between a position whereby the weir at least partially obstructs the pan during the start-up, shut-down and continuous modes, and a non-obstructive position after the shut-down sequence for discharge of coated articles from the pan.

6. The apparatus of claim 5, wherein said pan comprises a drum, and the weir comprises a segment of a cone mounted co-axially with the drum and which projects outwardly from the drum, said weir being mounted to the cabinet to remain stationary during rotation of the drum, and being adjustable about its axis to adjust its effective spill-over height.

7. The apparatus of claim 1 further comprising a cabinet for housing said pan, a cabinet support configured to selectively tilt said cabinet along the elongate axis of said pan, and an actuator associated with the cabinet support to effect said tilt, and wherein said controller is further configured to actuate said cabinet support to selectively distribute articles within the pan wherein the pan is levelled during at least part of the start up and shut down sequences, and declines towards the second end thereof during the continuous mode and optionally at the outset of the start up sequence and the termination of the shut-down sequence.

8. The apparatus of claim 1, wherein said means to distribute articles is provided by said pan comprising a drum journalled for rotation and drive means to rotate said drum, wherein distribution of tablets within the pan is obtained by rotation of the drum.

9. The apparatus of claim 1 further comprising a cabinet for housing said pan and an array of individually controllable heat sources for delivering heated gas into said cabinet and defining discrete heating zones disposed linearly along the length of said cabinet for drying and/or curing said articles, said heat sources being in operative communication with and responsive to said controller for selectively applying a heated airflow within selected ones of said zones.

10. The apparatus of claim 9 further comprising a plurality of bypass valves and bypass ducts associated with said heat sources and controlled by said controller whereby shutting off selected ones of said heat sources directs the airflow from said heat source into corresponding ones of said bypass ducts without entering into said cabinet.

11. A method for applying a coating to tablets or other articles in bulk, comprising the steps of:
a) providing an apparatus comprising an elongate pan for retaining a bed of said articles, said pan comprising opposed first and second ends wherein said articles are introduced into said pan at said first end and discharged from said second end, a coating delivery system for dispensing coating onto said articles in a plurality of individually-controlled spray zones located linearly along said pan, and a distribution system for distributing said articles along the pan wherein the full surface of all of said articles is exposed to said coating; and
b) selectively operating the apparatus in one of either i) a batch mode for a start-up or shut-down sequence wherein said coating delivery system is controlled to deliver different amounts of coating in respective ones of said zones to a bed of articles on said pan, wherein the amount of coating delivered in said start up sequence is selected to substantially fully coat only the articles located in the zone located at said second end and the amount of coating delivered in said shut down sequence is selected to substantially fully coat the full bed of said articles, and ii) a continuous mode wherein articles are introduced on a continuous basis into the first end of the pan and discharged from the second end of the pan while applying spray within all of said zones on a continuous basis.

12. The method of claim 11, wherein said delivery of different amounts of coating comprises delivering said spray for different durations within said zones.

13. The method of claim 12, wherein the start-up sequence comprises sequentially actuating said spray zones for delivery of spray for increasingly longer durations of coating delivery from the first end of the pan to the second end of the pan, and optionally providing a duration during which all of said spray zones are actuated; and wherein the shut-down sequence comprises sequentially actuating said spray zones for delivery of spray to provide increasingly shorter durations of coating delivery from the first end of the pan to the second end of the pan to fully coat all of the articles in the pan, followed by discharge of the articles from the pan.

14. (canceled)

15. The method of claim 11, wherein operating the apparatus in the start-up sequence comprises applying coating to an initial load of articles by:
a) activating said distribution system,
b) charging said pan with said initial load of articles,
c) distributing said articles to form a bed of generally even depth along said pan, and
d) discharging coating within all of said spay zones for a predetermined first duration to apply a partial layer of coating to the entirety of said initial load, and sequentially deactivating said spray zones commencing at the first end of the pan and terminating at said second end wherein said coating is applied for progressively longer durations from the first end to the second end and articles at the second end are fully coated.

16. The method of claim 11, wherein operating said apparatus in the shut-down sequence comprising stopping introduction of uncoated articles at the first end, sequentially deactivating said spray zones commencing at said second end and terminating at said first end, and discharging said articles from said second end.

17. The method of claim 12, wherein said spray zones are of equal length and said durations are equal for all of said zones for any one of a start up or shut down sequence.

18. The method of claim 11, wherein said apparatus further comprises a weir which may be adjusted in position relative to said pan between a first position for partially obstructing the second end of the said pan to maintain a selected depth of a bed of said articles within the pan and a second position which is non-obstructive, and a system for tilting said pan downwardly from said first end to said second end, and wherein said start up sequence comprises positioning said weir in said first position, introducing a charge of said articles into the first end of said pan, tilting said pan downwardly towards the second end to distribute said charge of articles to form a bed of generally even depth along said pan, followed by levelling said pan to the horizontal position while applying said spray, and wherein said continuous mode comprises tilting said pan downwardly while applying said spray, said downward tilt being selected to form a bed of generally even depth along said pan as said articles are continuously introduced at said first end and discharged at said second end.

19. The method of claim 18 further comprising a shut down sequence comprising stopping the introduction of articles into said pan from said continuous mode, levelling said pan, sequentially deactivating said spray zones commencing at said second end and terminating at said first end to fully coat all of the articles within the pan, and discharging said coated articles from the pan by positioning said weir in said second position and tilting said pan downwardly discharge all of said articles from said pan.

20. The method of claim 11, wherein said apparatus further comprises a cabinet housing said pan, an array of heat sources for delivering heated gas into said cabinet for drying and/or curing said articles within selected ones of said spray zones, and a plurality of bypass ducts and bypass valves associated with said heat sources, said method comprising the further steps of selectively controlling said heat sources for discharging heated gas within selected ones of said zones in coordination with the activation of liquid discharge within corresponding ones of said zones, and actuating said bypass valves to direct airflow from non-discharging ones of said heat sources into said bypass duct without entering into said cabinet.

21. (canceled)

22. An apparatus for applying a coating to tablets or other articles in bulk, comprising an elongate pan for retaining a bed of said articles, said pan comprising opposed first and second ends wherein said articles are introduced into said pan at said first end and discharged from said second end, a coating discharge system comprising a plurality of individually-controlled spray zones located linearly along said pan for dispensing coating onto said articles, a distribution system for distributing said articles along the pan wherein the full surface of all of said articles is exposed to said coating, and a controller comprising a program medium, wherein the controller is configured to control the apparatus according to the following sequence:
a) a batch mode start-up sequence to coat an initial load of articles comprising activating said distribution system, charging said pan with said initial load of articles, distributing said articles to form a bed of a generally even depth along said pan, discharging coating within all of said spay zones for a predetermined first duration to apply a partial layer of coating to the entirety of said initial load, and sequentially deactivating said spray zones commencing at the first end of the pan and terminating at said second end wherein said coating is applied for progressively longer durations from the first end to the second end and articles at the second end are fully coated; and
b) subsequently operating said apparatus in a continuous mode by introducing additional uncoated articles at the first end of said pan on a continuous basis, activating all of said spray zones, distributing said articles from the first end of the pan to the second end thereof, and discharging fully coated articles from the second end.

23. The apparatus of claim 22, wherein said controller is configured to provide the further step of operating said apparatus in a shut-down sequence comprising stopping the introduction of uncoated articles at the first end, sequentially deactivating said spray zones commencing at said second end and terminating at said first end, and discharging said articles from said second end.

24. The apparatus of claim 22, wherein said spray zones are of equal length and said controller is configured to provide sequential activation and deactivation of the coating discharge system within said zones which commences in activation in an initial zone followed by sequential activation in each subsequent zone after a predetermined duration from the activation of the immediately preceding activated zone, and said sequential deactivation commences in said initial zone followed by sequential deactivation in each subsequent zone after a predetermined duration from the deactivation of the immediately preceding deactivated zone, and wherein said durations are equal for all of said zones.

25. (canceled)

26. The apparatus of claim 22 further comprising a weir which may be adjusted in position relative to said pan between a first position for partially obstructing the second end of the said pan to maintain a selected depth of a bed of said articles within the pan and a second position which is non-obstructing, and a system for tilting said pan downwardly from said first end to said second end, and wherein said controller is configured to provide in the start up sequence the steps of: positioning said weir in said first position, introducing a charge of said articles into the first end of said pan, tilting said pan by a first downward degree selected to distribute said charge of said articles to form a bed of generally even depth along said pan, followed by levelling said pan to the horizontal position while applying said spray, and in the continuous mode the steps of: tilting said pan by downwardly while applying said spray, said downward tilt being selected to effect the formation of a bed of generally even depth along said pan as said articles are continuously introduced at said first end and discharged at said second end.

27. The apparatus of claim 26, wherein the controller is configured to provide a shut down sequence comprising stopping the introduction of articles into said pan from said continuous mode, levelling said pan, sequentially deactivating said spray zones commencing at said second end and terminating at said first end to fully coat all of the articles within the pan, and discharging said coated articles from the pan by positioning said weir in said second position and tilting said pan downwardly discharge all of said articles from said pan.

28. The apparatus of claim 22 further comprising a cabinet enclosing said pan, an array of heat sources for delivering heated gas into said cabinet for drying and/or curing said articles within selected ones of said spray zones, and a plurality of bypass ducts and bypass valves associated with said heat sources, wherein the controller is configured to provide the further steps of selectively controlling said heat sources for discharging heated gas within selected ones of said zones in coordination with the activation of liquid discharge within corresponding ones of said zones, and actuating said bypass valves to direct airflow from non-discharging ones of said heat sources into said bypass duct without entering into said cabinet.
